(19) United States
(12) Patent Application Publication
(10) Pub. No.: US 2022/0367012 A1
(43) Pub. Date: Nov. 17, 2022

(54) DIGITAL ASSISTANT TO SUPPORT PRODUCT DEVELOPMENT

(71) Applicant: BASF SE, Ludwigshafen (DE)

(72) Inventors: Ferdinand Paul Brandl, Ludwigshafen (DE); Thorsten Cech, Ludwigshafen (DE); Verena Geiselhart, Lampertheim (DE); Johann Philipp Hebestreit, Lampertheim (DE); Brett R. Burns, Florham Park, NJ (US); Andres Christian Orthofer, Lampertheim (DE); Christian Herbert Rueckold, Ludwigshafen (DE); Kai Sievert, Ludwigshafen (DE); Ulrike Hees, Ludwigshafen (DE); Felix Klug, Lampertheim (DE)

(21) Appl. No.: 17/624,417

(22) PCT Filed: Jul. 16, 2020
(86) PCT No.: PCT/EP2020/070176

§ 371 Date: Jan. 3, 2022

(30) Foreign Application Priority Data
Jul. 17, 2019 (EP) 19186818.1

(51) Int. Cl. G16C 20/50; G16C 20/30; G16H 70/40; G16C 20/20

(52) U.S. Cl. CPC G16C 20/50; G16C 20/30; G16H 70/40; G16C 20/20

(57) ABSTRACT

In order to facilitate product development, such as pharmaceutical product development, a computer implemented method and an apparatus are proposed that enable formulators to develop robust drug formulations in a cost- and time-efficient manner. To start the development process, the user selects the preferred dosage form (e.g., granules, pellets, capsules, tablets etc.), defines a target profile (e.g., amount of active ingredient per unit, size of dosage form, mechanical strength of dosage form, desired release behaviour etc.) and enters key characteristics of the active ingredient (e.g., true density, particle size distribution data, bulk and tapped density, angle of repose, compressibility and compactibility profile etc.). The identity (e.g., chemical name or structure) of the active ingredient is not necessarily disclosed. The apparatus processes the provided data and calculates key parameters of the AI (e.g., particle size, powder density, powder flow and tabletability) Similar key parameters are calculated for common pharmaceutical excipients and stored in the apparatus. The apparatus then selects all relevant excipients and suggests a suitable manufacturing process. Combinations of active ingredients and excipients qualify as drug formulation if the predicted properties comply with the defined target profile. The following aspects can be considered: solubility and permeability of the active ingredient, dissolution of the active ingredient, probability to pass the content uniformity criteria, flowability of the powder blend, tabletability of the powder blend, mechanical strength and size of the tablet, compatibility of active ingredients and excipients etc.

### FIELD OF THE INVENTION

The present invention relates to a computer-implemented method, an apparatus, and a system for identifying a suitable formulation for product development. The present invention further relates to a computer program element.

### BACKGROUND OF THE INVENTION

A good understanding of active ingredients, excipients and manufacturing processes is prerequisite for the development of high-quality products, such as drug products. Today, the development process is mostly experimental and driven by the expertise and intuition of the individual formulator. For example, drug product development is, therefore, often time-consuming and cost-intensive. Furthermore, there is a high risk of failure; in the worst case, the manufactured drug product may not comply with the current quality and regulatory standards. Similar considerations apply to the development of cleaning agents, cosmetic products, dietary supplements, fungicide formulations, herbicide formulations, pesticide formulations or washing agents.

### SUMMARY OF THE INVENTION

There may be a need to facilitate and support formulators during product development.

The object of the present invention is solved by the subject-matter of the independent claims, wherein further embodiments are incorporated in the dependent claims. It should be noted that the following described aspects of the invention apply also for the computer implemented method, the apparatus, the system, and the computer program element.

A first aspect of the present invention provides a computer implemented method for identifying a suitable formulation for product development, comprising:

a) receiving, via an input channel, a user input that defines:
a dosage form;
a target product profile, TPP, comprising a minimum product requirement; and
key physicochemical properties of an active ingredient, AI;
   b) calculating, by a processor, key parameters of the AI relevant for the development of the dosage form based on the key physicochemical properties of the AI;
   c) re-calculating, by the processor, the key parameters of the AI when combined with the one or more excipients selected from an excipient database;
   d) selecting, by the processor, at least one promising excipient from the one or more selected excipients capable of improving the key parameters of the AI;
   e) suggesting, by the processor, a manufacturing process based on the AI, the at least one selected promising excipient, and the dosage form;
   f) predicting, by the processor, product properties based on the suggested manufacturing process, a combination of the AI and the at least one selected promising excipient, and the dosage form;
   g) determining, by the processor, whether the predicted product properties comply with the user-defined TPP; and
   h) identifying, by the processor, a suitable formulation based on the combination of the AI and the at least one selected promising excipient, the suggested manufacturing process, and the dosage form, if it is determined that the predicted product properties comply with the user-defined TPP.

A further aspect of the present invention provides an apparatus for identifying a suitable formulation for product development, comprising:
an input unit: and
a processing unit configured for:
   a) receiving, via an input channel, a user input, via the input unit, wherein the user input defines:
a dosage form;
a target product profile, TPP, comprising a minimum product requirement; and
key physicochemical properties of an active ingredient, AI;
   b) calculate, by a processor, key parameters of the AI relevant for the development of the dosage form based on the key physicochemical properties of the AI;
   c) re-calculate, by the processor, the key parameters of the AI when combined with the one or more excipients selected from an excipient database;
   d) select, by the processor, at least one promising excipient from the one or more selected excipients capable of improving the key parameters of the AI;
   e) suggest, by the processor, a manufacturing process based on the AI, the at least one selected promising excipient, and the dosage form;
   f) predict, by the processor, product properties based on the suggested manufacturing process, a combination of the AI and the at least one selected promising excipient, and the dosage form;
   g) determine, by the processor, whether the predicted product properties comply with the user-defined TPP; and
   h) identify, by the processor, a suitable formulation based on the combination of the AI and the at least one selected promising excipient, the suggested manufacturing process, and the dosage form, if it is determined that the predicted product properties comply with the user-defined TPP.

In other words, a computer implemented method and an apparatus are proposed that enable formulators to develop robust drug formulations in a cost- and time-efficient manner. To start the development process, the user selects the preferred dosage form (e.g., granules, pellets, capsules, tablets etc.), defines a target profile (e.g., amount of active ingredient per unit, size of dosage form, mechanical strength of dosage form, desired release behaviour etc.) and enters key characteristics of the active ingredient (e.g., true density, particle size distribution data, bulk and tapped density, angle of repose, compressibility and compactibility profile etc.). The identity (e.g., chemical name or structure) of the active ingredient is not necessarily disclosed.

In step b), the apparatus processes the provided data and calculates key parameters of the AI (e.g., particle size, powder density, powder flow and tabletability) by normalizing and scaling the data. In other words, the apparatus evaluates the processability of an active ingredient without any excipients.

For example, fourteen parameters (i.e., dl 0 value, d50 value, d90 value, distribution span, bulk density, tapped density, compressibility index, Hausner ratio, angle of repose, compaction pressure at a porosity of 0.15, tensile strength at a porosity of 0.15, tensile strength at 100 MPa compaction pressure, tensile strength at 150 MPa compaction pressure and tensile strength at 250 MPa compaction pressure) may be used to evaluate the processability of the active ingredient. All parameters may be scaled from 0 to 10, where 0 means “insufficient” and 10 means “excellent”; 5 is the acceptance value for direct compression.

The parameters may be grouped into the following categories: processability (mean value of all parameters), particle size (mean value of d10 value, d50 value, d90 value and distribution span), powder density (mean value of bulk and tapped density), powder flow (mean value of compressibility index, Hausner ratio and angle of repose) and tabletability (mean value of compaction pressure at a porosity of 0.15, tensile strength at a porosity of 0.15, tensile strength at 100 MPa compaction pressure, tensile strength at 150 MPa compaction pressure and tensile strength at 250 MPa compaction pressure).

A radar chart may be used to quickly identify the strengths (values greater than or equal to 5) and weaknesses (values less than 5) of an active ingredient. In general, it can be concluded that the processability is improving, as the area circumscribed by the curve is increasing. Direct compression is possible if the parameters processability, powder flow and tabletability are greater than or equal to 5; dry granulation (e.g., roller compaction, slugging etc.) is possible if the tabletability parameter is greater than or equal to 5; wet granulation (e.g., fluid-bed or high-shear granulation) is always possible. An exemplary radar chart is shown in FIG. 3D.

In step c), the apparatus predicts key properties of the AI when combined with common pharmaceutical excipients by applying mixing rules; the apparatus subsequently processes the data and calculates key parameters of the AI when combined with common pharmaceutical excipients (e.g., particle size, powder density, powder flow and tabletability) by normalizing and scaling the data. In other words, the apparatus predicts the processability of powder blends (i.e., combinations of an active ingredient with a filler/binder). The user may need to enter the properties of the active ingredient, select an excipient or excipient blend from a list, and enter the weight fraction of the active ingredient.

For example, the properties of the powder blend (i.e., particle size distribution, bulk density, tapped density, angle of repose, compressibility and compactibility profile) are estimated from single-component data by applying mixing rules. To estimate the particle size distribution of the powder blend, the cumulative size distributions of the individual components are reconstructed from their d10, d50 and d90 values (it is assumed that the particles are spherical and log-normally distributed). The cumulative size distribution of the powder blend is then derived from the volume-weighted arithmetic mean of the individual curves.

For example, the mixing rules may use the following equation to predict the particle size distribution of the power blend:

$${F ( x )}_{mix} = {\sum }_{i} {y}_{i} {F ( x )}_{i}$$

in which F(x) is the cumulative distribution function of the particle size and yᵢ are the volume fractions of the components.

The bulk density of the powder blend is calculated from the weighted arithmetic mean of the individual values; more weight is given to the component with the smaller bulk density. The tapped density of the powder blend is calculated from the weighted arithmetic mean of the individual values; more weight is given to the component with the larger tapped density. The angle of repose of the powder blend is calculated from the weighted arithmetic mean of the individual values; more weight is given to the component with the larger angle of repose.

For example, the mixing rules may use the following equation to calculate the bulk density of the powder blend:

D_{bulk,mix}=(D_{bulk,1}·x₁·WF+D_{bulk,2}·x₂)/(x₁·WF+x₂)

in which D_{bulk,1} and D_{bulk,2} represent the smaller and larger bulk density, x₁ and x₂ represent the weight fractions of the components with smaller and larger bulk density, and WF is the particle size dependent weighting factor.

For example, the mixing rules may use the following equation to calculate the tapped density of the powder blend:

D_{tap,mix}=(D_{tap,1}·x₁+D_{tap,2}·x₂·WF)/(x₁+x₂·WF)

in which D_{tap,1} and D_{tap,2} represent the smaller and larger tapped density, x₁ and x₂ represent the weight fractions of the components with smaller and larger tapped density, and WF is the particle size dependent weighting factor.

A similar approach may be used to predict the angle of repose of the power blend.

For example, the mixing rules may use the following equation to calculate the angle of repose of the powder blend:

αₘᵢₓ=(α₁·x₁·WF+α₂·x₂)/(x₁·WF+x₂)

in which α₁ and α₂ represent the smaller and larger angle of repose, x₁ and x₂ represent the weight fractions of the components with larger and smaller angle of repose, and WF is the particle size dependent weighting factor.

The compressibility profile of the powder blend is derived from the volume-weighted arithmetic mean of the individual profiles; a least squares fitting is done to determine the compaction pressure at zero porosity and compressibility resistance.

For example, the mixing rules may use the following equation to calculate the compressibility profile of the powder blend:

$${ε ( P )}_{mix} = {\sum }_{i} {y}_{i} {ε ( P )}_{i}$$

in which ε(P) is the porosity of the tablet at compaction pressure P and yᵢ are the volume fractions of the components.

The compactibility profile of the powder blend may be derived from the volume-weighted geometric mean of the individual profiles; a least squares fitting may be done to determine the tensile strength at zero porosity and bonding capacity.

For example, the mixing rules may use the following equation to calculate the compactibility profile of the powder blend:

$${T}_{mix} = {\prod }_{i} {T ( {ε ( P )}_{i} )}_{i}^{{y}_{i}}$$

in which T(ε) is the tensile strength of the tablet at porosity ε and yᵢ are the volume fractions of the components.

In step d), the apparatus identifies suitable excipients.

For example, the apparatus calculates the weight fraction of the active ingredient based on the given dose and tablet weight; 7% are subtracted from the tablet weight to accommodate the disintegrant and lubricant. In the next step, the apparatus predicts the processability of all possible active ingredient-excipient combinations. The properties of the powder blends (i.e., particle size distribution, bulk density, tapped density, angle of repose, compressibility and compactibility profile) are estimated from single-component data by applying mixing rules.

Thus, fourteen parameters (i.e., d10 value, d50 value, d90 value, distribution span, bulk density, tapped density, compressibility index, Hausner ratio, angle of repose, compaction pressure at a porosity of 0.15, tensile strength at a porosity of 0.15, tensile strength at 100 MPa compaction pressure, tensile strength at 150 M Pa compaction pressure and tensile strength at 250 M Pa compaction pressure) may be used to evaluate the processability of the powder blends. All parameters may be scaled from 0 to 10, where 0 means “insufficient” and 10 means “excellent”; 5 is the acceptance value for direct compression.

The parameters may be grouped into the following categories: processability (mean value of all parameters), particle size (mean value of dl 0 value, d50 value, d90 value and distribution span), powder density (mean value of bulk and tapped density), powder flow (mean value of compressibility index, Hausner ratio and angle of repose) and tabletability (mean value of compaction pressure at a porosity of 0.15, tensile strength at a porosity of 0.15, tensile strength at 100 MPa compaction pressure, tensile strength at 150 MPa compaction pressure and tensile strength at 250 MPa compaction pressure).

The radar chart can also be used to quickly identify the strengths (values greater than or equal to 5) and weaknesses (values less than 5) of a powder blend. In general, it can be concluded that the processability is improving, as the area circumscribed by the curve is increasing. Direct compression is possible if the parameters processability, powder flow and tabletability are greater than or equal to 5; dry granulation (e.g., roller compaction, slugging etc.) is possible if the tabletability parameter is greater than or equal to 5; wet granulation (e.g., fluid-bed or high-shear granulation) is always possible.

Once all combinations have been tested, the apparatus sorts the tested excipients and excipient blends according to their performance. For this purpose, a weighted mean of all 14 parameters is calculated. More weight is given to tabletability and powder flow; particle size and powder density are less important.

In step e), the apparatus suggests a suitable manufacturing process and selects the most relevant excipient or excipient blend.

Using direct compression as an example, according to the Manufacturing Classification system, direct compression is feasible if the material meets certain requirements regarding particle size and shape, blend uniformity, powder flow, powder density, tableting performance, and mechanical strength of the compact.

For example, in direct compression processes, excipients and excipient blends with insufficient flowability and/or tabletability (values less than 5) are sorted out. All excipients and excipient blends are rated on a scale from zero (“not qualified”) to five stars (“most qualified”). “Five stars” denotes excipients or excipient blends with the highest performance; “one star” denotes excipients or excipient blends with the lowest performance; “zero stars” means that the excipient or excipient blend is not qualified for direct compression processes.

In step f), combinations of active ingredients and excipients qualify as drug formulation if the predicted product properties comply with the defined target profile. The following aspects can be considered: solubility and permeability of the active ingredient, dissolution of the active ingredient, probability to pass the content uniformity criteria, flowability of the powder blend, tabletability of the powder blend, mechanical strength and size of the tablet, compatibility of active ingredients and excipients etc.

Using the tabletability of the power blend as an example, the properties of the powder blend (i.e., compressibility and compactibility profile) are estimated from single-component data by applying mixing rules. The compressibility profile of the powder blend is derived from the volume-weighted arithmetic mean of the individual profiles; the compactibility profile of the powder blend is derived from the volume-weighted geometric mean of the individual profiles.

The compressibility describes the ability of the powder to undergo a reduction in volume as a result of the applied pressure; it is represented by a plot of porosity versus compaction pressure. The compactibility describes the ability of the powder to be transformed into tablets of specific strength during densification; it is represented by a plot of tensile strength versus porosity. The tabletability describes the ability of the powder to be transformed into tablets of specific strength as a result of the applied pressure; it is represented by a plot of tensile strength versus compaction pressure.

The compaction pressure is calculated from the applied force and the cross-sectional area of the punch tip. For cylindrical tablets, the compaction pressure can be calculated using the following equation:

P=4F/πD²

in which P is the compaction pressure, F is the applied force, and D is the tablet diameter. The porosity can be calculated from the tablet weight (m), the true density of the tablet (ρ), and the tablet volume (V):

ε=1−(m/pV)

The tensile strength provides a measure of the mechanical strength of a tablet considering its shape and dimensions. For cylindrical tablets, the tensile strength can be calculated using the following equation:

σ=2F/πDH

in which σ is the tensile strength, F is the breaking force, D is the tablet diameter, and H is the tablet thickness.

For example, by using the above described method, the apparatus may predict whether the required tensile strength of a tablet can be achieved within the provided compaction pressure limits.

In step h), the apparatus calculates a starting formulation with the selected filler-binder combination.

A superdisintegrant is added; the amount depends on the selected filler-binder combination. For example, if the filler-binder combination already contains a disintegrant (e.g., ready-to-use excipients), the amount of superdisintegrant is reduced accordingly. If the tabletability of the powder blend is medium (value less than 6), a superdisintegrant with binding properties is chosen (e.g., Kollidon® CL-SF); otherwise, a regular superdisintegrant (e.g., Kollidon® CL-F) is added. Sodium stearyl fumarate is added as a lubricant; the amount depends on the selected filler-binder combination. In contrast to magnesium stearate, sodium stearyl fumarate does not cause overlubrication; it shows less incompatibilities with active ingredients. Once the correct amounts of superdisintegrant and lubricant have been determined, the amount of the filler-binder combination is recalculated.

As will be explained hereafter and particularly with respect to the exemplary embodiment in FIG. 4, if no formulation has been identified, the apparatus may suggest additional technological measures (e.g., milling or micronization, addition of and processing with excipients etc.) to optimize the characteristics of the active ingredient. Alternatively, the apparatus may suggest adjusting the target profile or selecting a different dosage form. In the next step, the user may re-enter the key characteristics of the active ingredient, re-define the target profile or select a different dosage form. The development process may start over and the apparatus may re-evaluate the data. If at least one formulation has been identified, the user prepares the formulation in the laboratory and characterizes the obtained product. The development process is finished if the experimental results (e.g., content uniformity, dissolution profile, mechanical strength of tablet etc.) comply with the target profile. Otherwise, the apparatus suggests optimizing the properties of the active ingredient, adjusting the target profile or selecting a different dosage form. In the last step, the user has the possibility to print the formulation, download relevant information (e.g., quality, regulatory and technical documents) and order product samples. In addition, the user has the possibility to provide feedback (e.g., regarding usability, information content, formulation outcome etc.), which is used to improve the user experience and the apparatus itself. The request from authorities to pursue a quality-by-design approach is ideally realized by using this apparatus. Besides conventional tablet formulations, the apparatus may also support the development of other dosage forms such as a capsule including a hard capsule and a soft capsule, a chewing gum, a cream, an emulsion including an emulsion concentrate and a microemulsion, a foam, a spray, a gel, a stick, granules, gummies, an implant, an ointment, a paste, pellets including coated pellets, a powder, a solution including an injection solution, a suppository, a suspension including a suspension concentrate, a sustained-release form, a tablet including a buccal tablet, a chewable tablet, a coated tablet, a detergent tablet, a dishwashing tablet, an effervescent tablet, a lozenge, an orodispersible tablet, and a vaginal tablet, and a therapeutic patch.

Manufacturing technologies covered by the apparatus include coating, direct compression, granulation, extrusion, pelletizing, 3D-printing, emulsification, dispersion, blending, dissolving, encapsulation, prilling, roller compaction and various drying technologies such as spray drying and other formulation technologies. These technologies can be applied in a batch mode or continuously. The active ingredients can be selected from the group of pharmaceutical, nutritional, cosmetic, agrochemical or washing agents.

This may help the users (e.g., a business) to identify suitable formulations. The number of lab experiments would be reduced to an absolute minimum. This would speed up formulation development and save cost.

According to an embodiment of the present invention, if it is determined that the predicted product properties do not comply with the user-defined TPP or if it is determined that an experimental result obtained after preparing and characterizing the identified suitable formulation does not comply with the user defined TPP, the method further comprises performing at least one of the following steps: suggesting at least one additional technological measure to optimize the key parameters of the AI, based on a difference between the predicted product properties and the user-defined TPP or a difference between the experimental result and the user-defined TPP; suggesting to adjust the user-defined TPP based on a difference between the predicted product properties and the user-defined TPP or a difference between the experimental result and the user-defined TPP; and suggesting to select a different dosage form based on a difference between the predicted product properties and the user-defined TPP or a difference between the experimental result and the user-defined TPP.

In other words, if no formulation has been identified, additional technological measures (e.g., milling or micronization, addition of and processing with excipients etc.) are suggested to optimize the characteristics of the active ingredient. Alternatively or additionally, it may be suggested to adjust the target profile or select a different dosage form. The same applies when the experimental results do not comply with the target profile. For example, if at least one formulation has been identified, the user prepares the formulation in the laboratory and characterizes the obtained product. The development process is finished if the experimental results (e.g., content uniformity, dissolution profile, mechanical strength of tablet, etc.) comply with the target profile. Otherwise, the system may suggest optimizing the properties of the AI, adjusting the target profile, and/or selecting a different dosage form.

According to an embodiment of the present invention, the at least one additional technical measure comprises at least one of: milling or micronization, and addition of and processing with excipients.

According to an embodiment of the present invention, if the predicted product properties do not comply with the user-defined TPP or if it is determined that the experimental result obtained after preparing and characterizing the identified suitable formulation does not comply with the user defined TPP, the method further comprises repeatedly performing a sequence comprising: receiving a further user input related to a different dosage form, a user-redefined TPP, and/or re-determined key physicochemical properties of the AI; and performing steps b) to h), until a suitable formulation has been identified with the product properties complying with the user-defined or user-redefined TPP.

According to an embodiment of the present invention, the product development comprises at least one of the following: development of cleaning agents, development of cosmetic products, development of dietary supplements, development of drug products, development of fungicide formulations, development of herbicide formulations, development of pesticide formulations, and development of washing agents.

According to an embodiment of the present invention, the dosage form comprises at least one of a capsule including a hard capsule and a soft capsule, a chewing gum, a cream, an emulsion including an emulsion concentrate and a microemulsion, a foam, a spray, a gel, a stick, granules, gummies, an implant, an ointment, a paste, pellets including coated pellets, a powder, a solution including an injection solution, a suppository, a suspension including a suspension concentrate, a sustained-release form, a tablet including a buccal tablet, a chewable tablet, a coated tablet, a detergent tablet, a dishwashing tablet, an effervescent tablet, a lozenge, an orodispersible tablet, and a vaginal tablet, and a therapeutic patch.

A user interface (e.g., command line, graphical user interface) may be provided to facilitate the user to select one of the dosage forms.

According to an embodiment of the present invention, the user-defined TPP comprises at least one of: amount and/or concentration of the active ingredient; size, volume and/or weight of the dosage form; mechanical and/or rheological properties of the dosage form; release profile of the active ingredient; other application-relevant parameters; compatibility and stability; and other manufacturing-relevant properties.

These are general examples of the user-defined TPP applicable to all kinds of dosage forms. A user interface (e.g., command line, graphical user interface) may be provided to facilitate the user to define the TPP. This will be explained hereafter and particularly with respect to the exemplary embodiments in FIGS. 2 and 3C.

According to an embodiment of the present invention, the user-defined TPP comprises at least one of: amount of AI per unit; size and/or weight of the dosage form; mechanical strength of the dosage form; desired release behaviour of the dosage form; disintegration time of the dosage form; dissolution profile of the AI; compatibility of active ingredients and excipients; probability to pass content uniformity criteria; flowability of a powder blend; tabletability of a powder blend; and compatibility and stability of active ingredients and excipients.

The above-mentioned user-defined TPP is particularly applicable to solid oral dosage forms.

According to an embodiment of the present invention, the user-defined TPP comprises at least one of: concentration of AI; volume of the dosage form; rheological behaviour and/or viscosity of the dosage form; spreading and/or adherence of the dosage form; dispersity and/or volume fractions of phases; hydrophilicity and/or lipophilicity; release behaviour of the dosage form; melting point of the dosage form; dissolution profile of the AI; and compatibility and stability of active ingredients and excipients.

The above-mentioned user-defined TPP is particularly applicable to liquid and semi-solid dosage forms.

According to an embodiment of the present invention, the key physicochemical properties of the AI comprise at least one of: hydrophilicity and/or lipophilicity (e.g., distribution coefficient); melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactibility; hygroscopicity; water content (e.g., loss on drying); concentration of impurities; other chemical, physicochemical and/or physical properties; and information on compatibility and stability.

A user may select an AI from an AI database; the key physiochemical properties of the selected AI are also obtained from the AI database automatically. Alternatively, the user may define via a user interface the key physiochemical properties of the AI. This will be explained hereafter and particularly with respect to the exemplary embodiments in FIGS. 2 and 3B.

According to an embodiment of the present invention, the user-defined TPP comprises a dose of AI per unit and a maximum weight of the dosage form. Step c) further comprises the steps of calculating weight fractions of the AI and the one or more excipients selected from the excipient database based on the dose of AI per unit and the maximum weight of the dosage form and predicting properties of a combination of the AI and the one or more excipients. Step d) further comprises selecting at least one promising excipient from the one or more excipients if the properties of a corresponding mixture satisfy a predefined criterion.

In other words, it is proposed to expect the dose of the drug and the tablet weight as input parameters. The weight fractions of the drug and the excipient are calculated. The properties of all potential drug-excipient mixtures are predicted. Excipients are recommended if e.g. flowability and tabletability of the corresponding mixtures satisfy a predefined criterion, e.g. greater than or equal to 5, when all parameters are scaled from 0 to 10, where 0 means “insufficient” and 10 means “excellent”; 5 is the acceptance value.

As the dose of the drug and tablet weight are specified, the size of e.g. tablets can be controlled within a suitable range.

According to an embodiment of the present invention, the dosage form comprises a pharmaceutical dosage form.

In an example, the processing unit is further configured to allow a user to: print the formulation, download relevant information including quality information, regulatory information, safety data, and/or technical documents, order product samples, and/or provide a user-feedback including usability, information content and/or formulation outcome.

A further aspect of the present invention provides a system for identifying a suitable formulation for product development. The system comprises an apparatus according to any one of the above and below described exemplary embodiments and examples. The system further comprises a web server configured to interface with a user via a webpage and/or an application program served by the web server. The apparatus is configured to provide a graphical user interface, GUI, to a user, by the webpage and/or the application program.

A further aspect of the present invention provides a computer program element comprising sets of instructions, wherein, when the sets of instructions are executed on a processor of the apparatus of any one of the above and below described exemplary embodiments and examples, the sets of instructions cause the apparatus or the system to perform the method of any one of the above and below described exemplary embodiments and examples.

### BRIEF DESCRIPTION OF THE DRAWINGS

These and other aspects of the invention will be apparent from and elucidated further with reference to the embodiments described by way of examples in the following description and with reference to the accompanying drawings, in which

FIG. 1 is a block diagram of an example system for identifying a suitable formulation for product development.

FIG. 2 is a block diagram of an example apparatus for identifying a suitable formulation for product development.

FIG. 3A-31 illustrate an example of graphical user interfaces that provide assistance for product development.

FIG. 4 is a flowchart representative of example machine readable instructions for implementing the apparatus of FIGS. 1 and/or 2.

It should be noted that the figures are purely diagrammatic and not drawn to scale. In the figures, elements which correspond to elements already described may have the same reference numerals. Examples, embodiments or optional features, whether indicated as non-limiting or not, are not to be understood as limiting the invention as claimed.

### DETAILED DESCRIPTION OF EMBODIMENTS

To support formulators during product development, a digital assistant is proposed that shortens the development time by avoiding unnecessary lab experiments, lowers the risk of failure and ensures compliance with the quality target product profile. In particular, a science-based formulation prediction system is proposed, which enables formulators to develop more robust drug formulations. To retain confidentiality, the disclosed apparatuses, systems, and methods do not require the formulator to disclose the identity or structure of the active ingredient. Instead, it is proposed to predict the processability of hypothetical drug-excipient mixtures, and calculate a starting formulation depending on the selected dosage form and defined target profile. Disclosed apparatuses, systems, and methods may have a benefit that is that the number of time-consuming and costly lab experiments can be reduced. Disclosed apparatuses, systems, and methods may improve the efficacy and reduce the risk at early stages of formulation development.

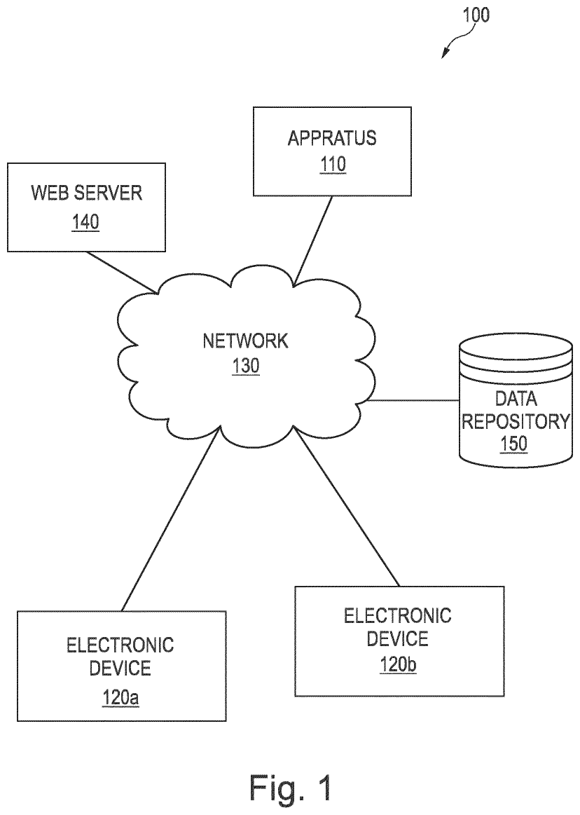

FIG. 1 is a block diagram of an example system 100 for identifying a suitable formulation for product development. Examples of the product development may include, but not limited to, development of dietary supplements, development of cosmetic products, development of fungicide, herbicide and/or pesticide formulations, development of cleaning and/or washing agents, and drug product development. The example system 100 of FIG. 1 includes an apparatus 110 for identifying a suitable formulation for product development, one or more electronic devices 120 (e.g., a first electronic device 120a, a second electronic device 120b), a network 130, a web server 140, and a data repository 150.

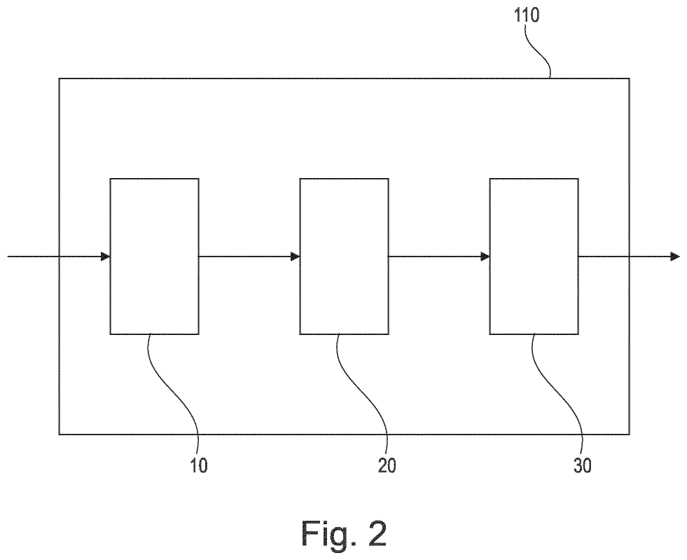

The example apparatus 110 of FIG. 1 is a computing device having processing capabilities for identifying a suitable formulation based on a user input that defines a dosage form, a TTP, and key physicochemical properties of an AI. The example apparatus 110 of the illustrated example may be a server that communicates with the example electronic devices 120 to authenticate users of the example electronic devices 120 to provide a user input including a dosage form, a TPP that comprises a minimum product requirement, and key physicochemical properties of an AI, and to transmit a suitable formulation derived by the example apparatus 110 to the example electronic devices 120. The apparatus 110 may be integrated with other components of the system 100 (e.g., the web server 140 and/or the data repository 150). An example implementation of the apparatus 110 will be described in conjunction with FIG. 2.

The electronic devices 120 of the illustrate example are used by a user (e.g., a business interested in product development) in the system 100 to communicate with the example apparatus 110 to input parameters (e.g., dosage form, TPP, key physicochemical properties of an AI) and to obtain an analysis result whether these input parameters can produce a product with product properties that comply with the user-defined TPP. If it is determined that the product properties comply with the user-defined TPP, the user may further obtain a suitable formulation derived by the apparatus 110. The electronic devices 120 of the illustrated example of FIG. 1 may be a mobile device, such as a cellular telephone. Alternatively, the electronic device may be any type of electronic device that is capable of communicating with the apparatus 110 to develop the product formulation. Further examples of the electronic devices 120 may be a personal computer (PC), or a workstation. The electronic devices 120 may be a computing device. Alternatively, the electronic devices 120 may be a device that typically does not include processing capabilities. For example, the electronic device 120 may be a simple user interface, e.g., touch screen, allowing a user to input parameters. The electronic device 120a, 120b may have a processor, which has been added to connect the device to a network. Examples of the electronic devices 120a, 120b may include mobile devices and PCs.

The network 130 of the illustrated example communicatively couples the example apparatus 110, the example web server 140, the example data repository 150, and the one or more electronic devices 120. The example network 130 is the internet. The network 130 may alternatively be any other type and number of networks. For example, the network 130 may be implemented by several local area networks connected to a wide area network. For example, the network 130 may comprise any combination of wired networks, wireless networks, wide area networks, local area networks, etc.

The example web server 140 of FIG. 1 is a server that provides a web service to facilitate a user of the one or more electronic devices 120 to access the example apparatus 110 for identifying a suitable formulation for their product development. The example web server 140 may include an interface through which a user can authenticate, e.g., by providing a username and password; an interface for creating, modifying, and deleting user accounts for the example apparatus 110; and an interface for creating, modifying, and deleting development projects for different product developments.

The example web server 140 may interface with the users via webpages and/or application programs served by the example web server 140 to facilitate the management of his development projects using the example apparatus 110. The example web server 140 may, alternatively, be replaced with another device (e.g., another computing device) that provide any type of interface (e.g., a command line interface, a graphical user interface, etc.). In some systems, the apparatus 110 may include an integrated web server unit and the separate webserver 140 may be included.

The example data repository 150 of FIG. 1 is a datastore that stores data including, for example, an AI database and an excipients database. Furthermore, the datastore can contain examples of final products including their recipes, manufacturing technologies and properties. The data repository 150 may be any type of datastore including a server, a database, a file, etc. While the example system 100 includes the example data repository 150, some implementations of the system 100 may not include the data repository 150. For example, the functionality of the data repository 150 may be integrated in the example apparatus 110.

FIG. 2 is a block diagram of an example implementation of the apparatus 110 of FIG. 1 for identifying a suitable formulation for product development. The product development may comprise at least one of the following: development of dietary supplements, development of cosmetic products, development of fungicide, herbicide and/or pesticide formulations, development of cleaning and/or washing agents, and drug product development. In the following, the discussion is focused on a pharmaceutical product development. However, a skilled person will appreciate that the following discussion may also be applied to other product development, such as development of cosmetic products.

The example apparatus 110 of FIG. 2 includes an example input unit 10, an example processing unit 20, and an example output unit 30. While the example apparatus 110 may be a server, the apparatus may, alternatively, be any other type of computing device (e.g., a desktop computer, a laptop computer, etc.).

The example input unit 10 is configured to receive a user input e.g. from one or more example electronic devices 120 of FIG. 1. The user input defines a dosage form, a TTP, and key physicochemical properties of an AI.

Examples of the dosage form may include, but not limited to, a capsule including a hard capsule and a soft capsule, a chewing gum, a cream, an emulsion including an emulsion concentrate and a microemulsion, a foam, a gel, granules, gummies, an implant, an ointment, a paste, pellets including coated pellets, a powder, a solution including an injection solution, a suppository, a suspension including a suspension concentrate, a sustained-release form, a tablet including a buccal tablet, a chewable tablet, a coated tablet, a detergent tablet, a dishwashing tablet, an effervescent tablet, a lozenge, an orodispersible tablet, and a vaginal tablet, and a therapeutic patch.

General examples of the user-defined TPP applicable to all kinds of dosage forms may include, but not limited to, amount and/or concentration of the active ingredient; size, volume and/or weight of the dosage form; mechanical and/or rheological properties of the dosage form; release profile of the active ingredient; other application-relevant parameters; compatibility and stability; and other manufacturing-relevant properties.

Particular examples of the user-defined TPP for solid oral dosage forms may include, but not limited to, amount of AI per unit; size and/or weight of the dosage form; mechanical strength of the dosage form; desired release behaviour of the dosage form; disintegration time of the dosage form; dissolution profile of the AI; compatibility of active ingredients and excipients; probability to pass content uniformity criteria; flowability of a powder blend; tabletability of a powder blend; and compatibility and stability of active ingredients and excipients.

Particular examples of the user-defined TPP for liquid and semi-solid dosage forms may include, but not limited to, concentration of AI; volume of the dosage form; rheological behaviour and/or viscosity of the dosage form; spreading and/or adherence of the dosage form; dispersity and/or volume fractions of phases; hydrophilicity and/or lipophilicity; release behaviour of the dosage form; melting point of the dosage form; dissolution profile of the AI; and compatibility and stability of active ingredients and excipients.

The key physicochemical properties of the AI may comprise at least one of: hydrophilicity and/or lipophilicity (e.g., distribution coefficient); melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactibility; hygroscopicity; water content (e.g., loss on drying); concentration of impurities; other chemical, physicochemical and/or physical properties; and information on compatibility and stability.

To facilitate a user to select and define these parameters, the example web server 140 of FIG. 1 may be configured to interface with a user via a webpage and/or an application program served by the web server. The webpage and/or the application program presents graphical user interfaces on a display of the example electronic devices 120 to allow the user to select and define these parameters. For example, the webpage and/or the application program may provide a first graphical user interface for receiving user credentials for authentication, a second graphical interface for allowing a user to select a desired dosage form a list of dosage forms, a third graphical user interface for allowing a user to select and define desired parameters among a list of TPPs, and a fourth graphical user interface for allowing to select and define the key physicochemical properties of the AI among a list of physicochemical properties of the AI. To facilitate the user to define the relevant parameters, the graphical user interfaces may highlight certain important parameters that are relevant for the selected dosage form.

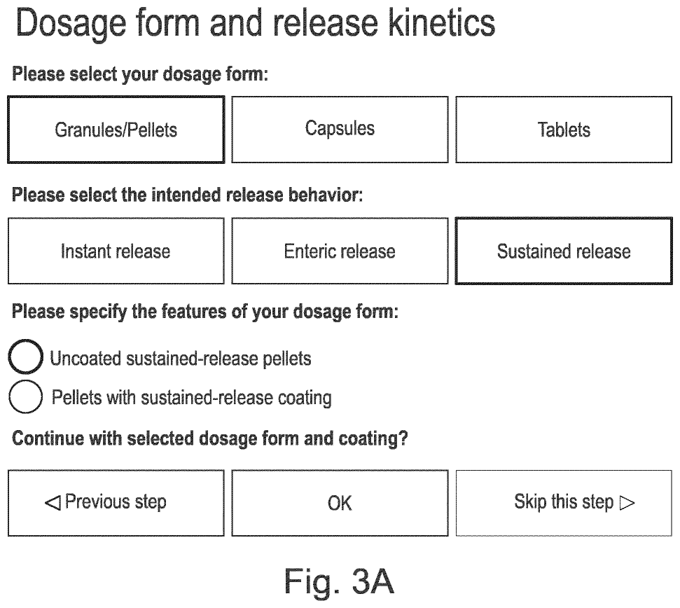

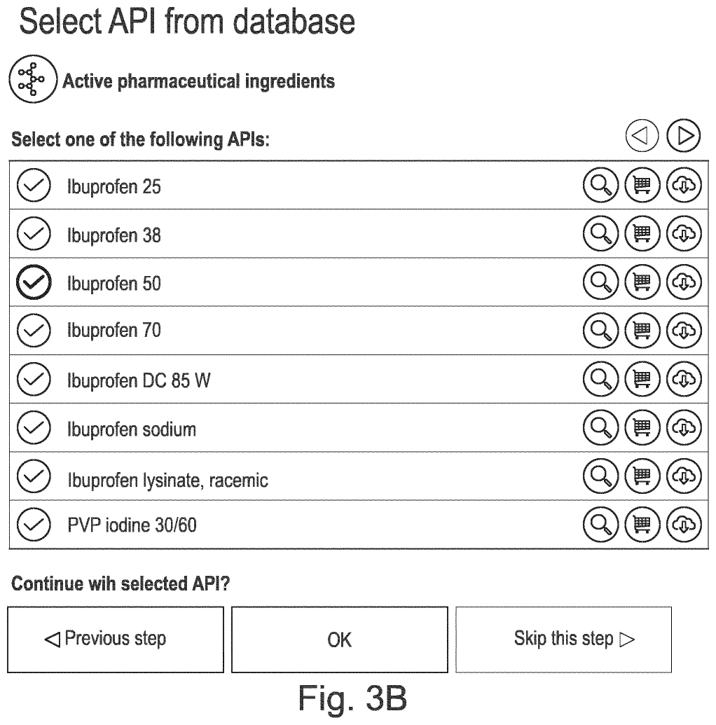

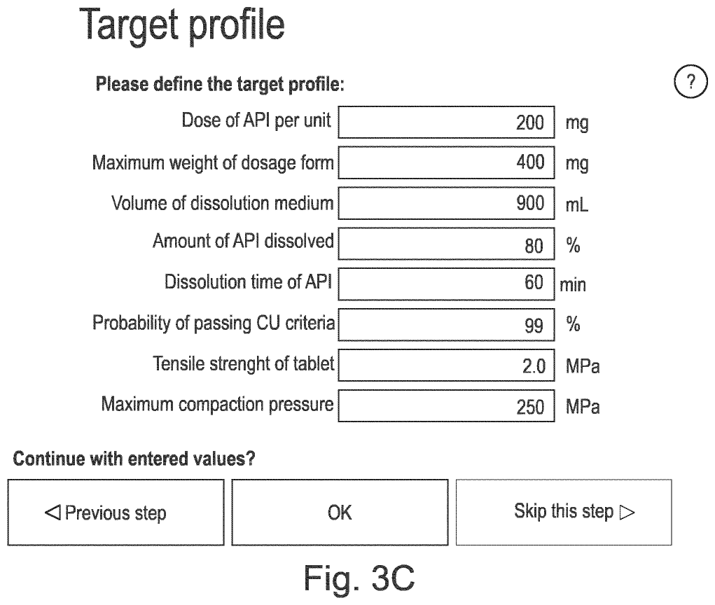

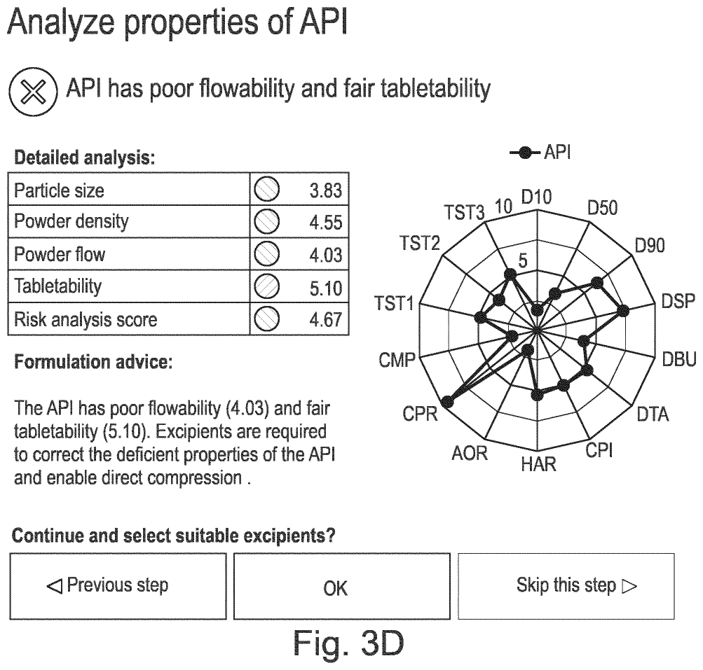

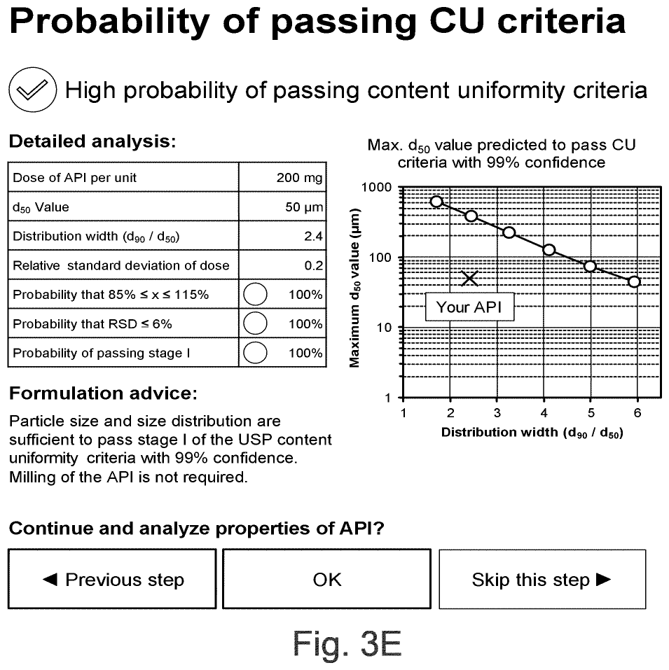

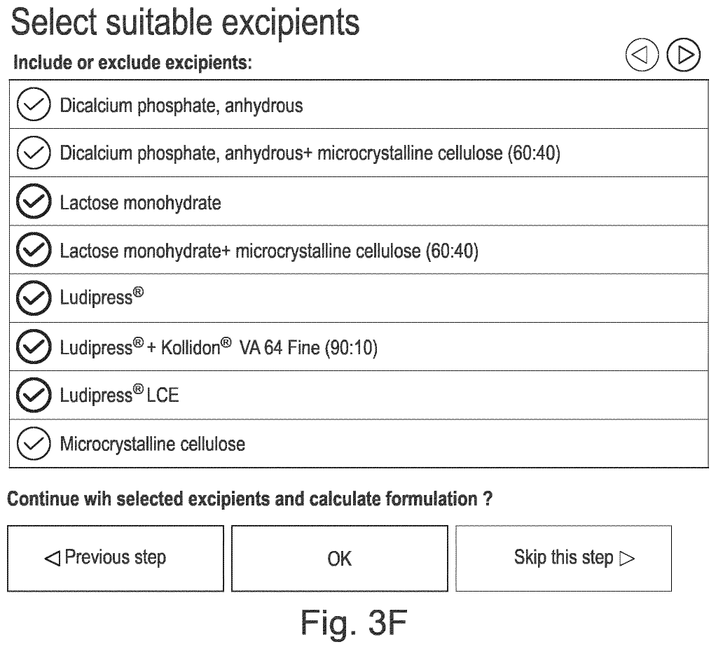

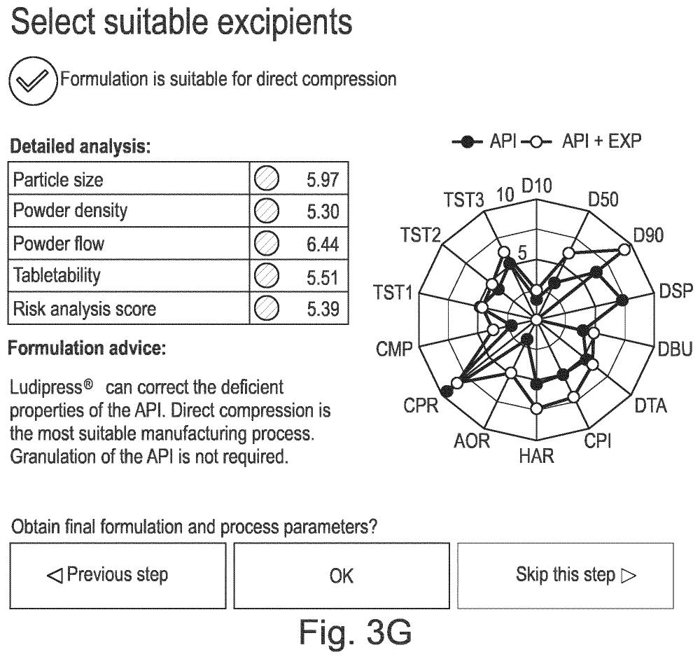

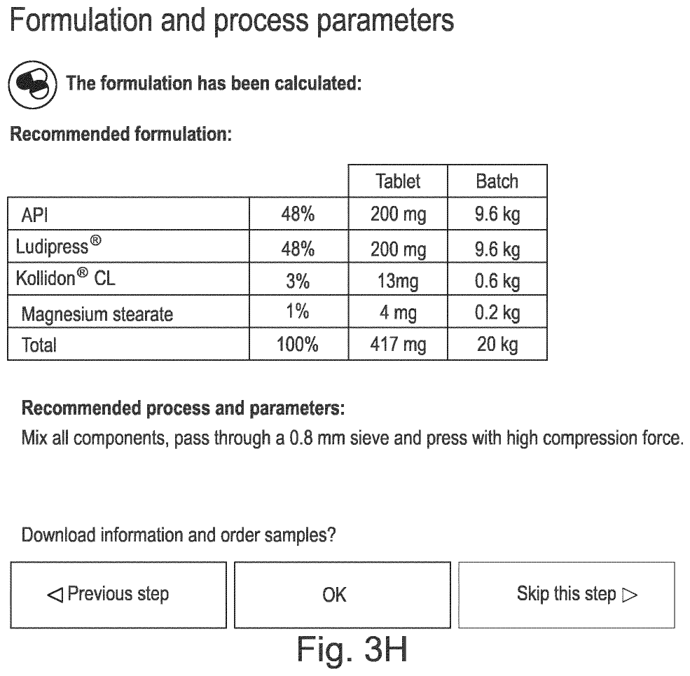

An example implementation of the second graphical user interface is described in conjunction with FIG. 3A. The second graphical user interface of the illustrated example allows the user to select at least one of the following dosage forms: granules/pellets, capsules, and tablets. The second graphical user interface of the illustrated example also allows the user to select the intended release behaviour, such as instant release, enteric release, and sustained release. The second graphical user interface of the illustrated example also allows the user to specify the features of the dosage form, such as conventional granules, conventional pellets, effervescent granules, etc.

An example implementation of the third graphical user interface is described in conjunction with FIG. 3B. The third graphical user interface of the illustrated example in FIG. 3B allows the user to select an AI from an AI database. In the illustrated example, the AI is related to active pharmaceutical ingredients (API). Therefore, in FIG. 3B, the term “API” is used instead of AI for indicating that the AI is related for pharmaceutical product development. Alternatively, the third graphical user interface may also allow the user to enter the properties of the API, such as d₁₀ value, d₅₀ value, d₉₀ value, bulk density, tapped density, angle of repose, true density, compaction pressure required to produce a compact of zero porosity, compressibility resistance, tensile strength at zero porosity, bonding capacity, etc.

An example implementation of the fourth graphical user interface is described in conjunction with FIG. 3C. The fourth graphical user interface allows the user to define dose of API per unit, maximum weight of dosage form, volume of dissolution medium, amount of API dissolved, dissolution time of API, probability of passing content uniformity criteria, tensile strength of tablet, and maximum compaction pressure.

Once the user input is received, the example processing unit 20 is configured to calculate key parameters of the AI relevant for the development of the dosage form based on the key physicochemical properties of the AI, which is selected from the AI database, or defined by the user. The key parameters of the AI are calculated from the key physicochemical properties of the AI by normalizing and scaling the data. The webpage and/or the application program may provide a fifth graphical user interface for allowing the user to view the analysis result.

An example implementation of the fifth graphical user interface is described in conjunction with FIG. 3D. The fifth graphical user interface of the illustrated example shows in the radar chart the parameters of the AI calculated from the key physicochemical properties of the API by normalizing and scaling the data, such as d₁₀ value (D10), d₅₀ value (D50), d₉₀ value (D90), distribution span (DSP), bulk density (DBU), tapped density (DTA), compressibility index (CPI), Hausner ratio (HAR), angle of repose (AOR), compressibility (CPR), compactibility (CMP), tensile strength (TST), etc. Based on these parameters, four key parameters (i.e., particle size, powder density, powder flow, and tabletability) and the risk analysis score are determined. The risk analysis score indicates the processability of the AI using a specified manufacturing technology (e.g., direct compression into tablets). In the illustrated example in FIG. 3D, it is indicated that the API has poor flowability and fair tabletability, and excipients are required to correct the deficient properties of the API.

The processing unit 20 of the illustrated example in FIG. 2 is also configured to re-calculate the key parameters of the AIs when combined with the one or more excipients selected from an excipient database. The desired excipient is the one that can correct the deficient properties of the API, that is, the one capable of improving the flowability and tabletability of the illustrated example in FIG. 3D. The outcome of the development process not only depends on the properties and quality of the AI, but also on the careful selection of excipients. The selection of excipients is based on multiple criteria including availability, compatibility, functionality, price, processability, safety, stability, quality, and regulatory aspects. Knowledge of relevant drug-excipient combinations is therefore very useful to the formulator in selecting appropriate excipients. This information may already be in existence for and stored in an excipient database and/or AI database. The webpage and/or the application program may provide a sixth graphical user interface for allowing the user to select an excipient from an excipient database.

Besides excipients capable of improving the flowability (e.g., fillers, fillers/binders, glidants, etc.) and tabletability (e.g., binders, fillers, filler/binders, etc.) of the AI, further excipients capable of improving the manufacturability of the AI (e.g., lubricants), improving the disintegration time of the tablet (e.g., disintegrants), increasing the solubility (e.g., surfactants, wetting agents, etc.) of the AI, or modifying the release behaviour of the tablet may be considered.

An example implementation of the sixth graphical user interface is described in conjunction with FIG. 3F. The sixth graphical user interface of the illustrated example allows the user to select one or more excipients from an excipient database store in the example data repository 150 in FIG. 1. Alternatively, the apparatus 110 may automatically select all the excipients in the excipient database. The apparatus 110 then re-calculates the parameters of the AI when combined with the one or more selected excipients based on pre-established mixing rules. These mixing rules are unique for each of the parameters and stored in the apparatus 110.

The processing unit 20 of the illustrated example in FIG. 2 is further configured to select at least one promising excipient from the one or more selected excipients capable of improving the key parameters of the AI. The processing unit 20 may rank the excipients based on their improvements on the key parameters and select one or more top ranked excipients as promising excipients. The webpage and/or the application program may provide a seventh graphical user interface for allowing the user to select an excipient from an excipient database.

An example implementation of the seventh graphical user interface is described in conjunction with FIG. 3G. The seventh graphical user interface of the illustrated example allows the user to view the improvement of the key parameters in the radar chart. The deficiency in the four key parameters (i.e., particle size, powder density, powder flow, and tabletability) has also been corrected, and the risk analysis score has been improved.

The processing unit 20 is also configured to suggest a manufacturing process based on the AI, the at least one selected promising excipient, and the dosage form. Knowledge on relevant drug-excipient combinations and corresponding manufacturing processes may already be in existence and stored in an excipient database and/or an AI database. The process unit 20 may extract this information from the excipient database and/or the AI database and provide the suggestion. The risk analysis score indicates the processability of the AI when combined with the at least one selected promising excipient using the suggested manufacturing technology.

For example, in FIG. 3G, it is recommended to use direct compression as the most suitable manufacturing process.

The processing unit 20 is further configured to predict product properties based on the suggested manufacturing process, a combination of the AI and the at least one selected promising excipient, and the dosage form, and to determine whether the predicted product properties comply with the user-defined TPP.

The processing unit 20 is further configured to identify a suitable formulation based on the combination of the AI and the at least one selected promising excipient, the suggested manufacturing process, and the dosage form, if it is determined that the predicted product properties comply with the user-defined TPP. Besides excipients capable of improving the flowability (e.g., fillers, fillers/binders, glidants, etc.) and tabletability (e.g., binders, fillers, filler/binders, etc.) of the AI, further excipients capable of improving the manufacturability of the AI (e.g., lubricants), improving the disintegration time of the tablet (e.g., disintegrants), increasing the solubility (e.g., surfactants, wetting agents, etc.) of the AI, or modifying the release behaviour of the tablet may be included. Knowledge on relevant drug-excipient combinations and corresponding manufacturing processes may already be in existence and stored in an excipient database and/or an AI database. The webpage and/or the application program may provide an eighth graphical user interface for allowing the user to review the identified formulation.

An example implementation of the eighth graphical user interface is described in conjunction with FIG. 3H. The eight graphical user interface of the illustrated example allows the user to view the components used in the formulation and the corresponding percentages. The formulation of the illustrated example in FIG. 3H has 48% of API, 48% of Ludipress®, 3% of Kollidon® CL, and 1% of magnesium stearate. The suggested manufacturing process and related parameters may also be presented to the user to facilitate the user to develop the product.

However, if it is determined that the predicted product properties do not comply with the user-defined TPP, the processing unit 20 may be configured to perform at least one of the following steps: suggesting at least one additional technological measure to optimize the key parameters of the AI, based on a difference between the predicted product properties and the user-defined TPP; suggesting to adjust the user-defined TPP based on a difference between the predicted product properties and the user-defined TPP; and suggesting to select a different dosage form based on a difference between the predicted product properties and the user-defined TPP. The at least one additional technical measure comprises at least one of: milling or micronization, and addition of and processing with excipients. The processing unit 20 may be configured to repeatedly performing a sequence comprising receiving a further user input related to a different dosage form, a user-redefined TPP, and/or re-determined key physicochemical properties of the AI; and performing the steps in the above described embodiments and example in FIG. 3A to 3H, until a suitable formulation has been identified with the product properties complying with the user-defined or user-redefined TPP.

As an option, the processing unit 20 is further configured to allow a user to order product samples, such as the promising excipients and/or other components used for the formulation, to print the formulation, and/or download relevant information including quality information, regulatory information, safety data, and/or technical documents. The webpage and/or the application program may provide a ninth graphical user interface for allowing the user to select and order an excipient from an excipient database. An example implementation of the ninth graphical user interface is described in conjunction with FIG. 3I. The ninth graphical user interface allows the user to select the Ludipress® and/or the Kollidon® CL used in the formulation. As a further option, a further graphical user interface may be provided to allow the user to provide feedback including, e.g., usability, information content, and/or formulation outcome.

The apparatus 110 may further comprise an output unit for outputting the analysis results.

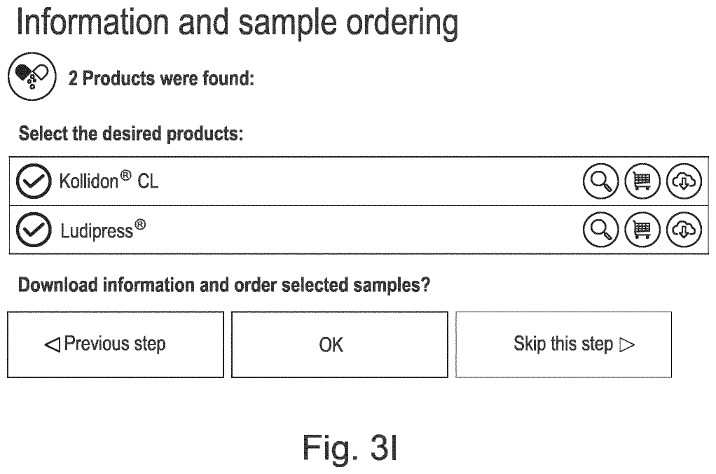

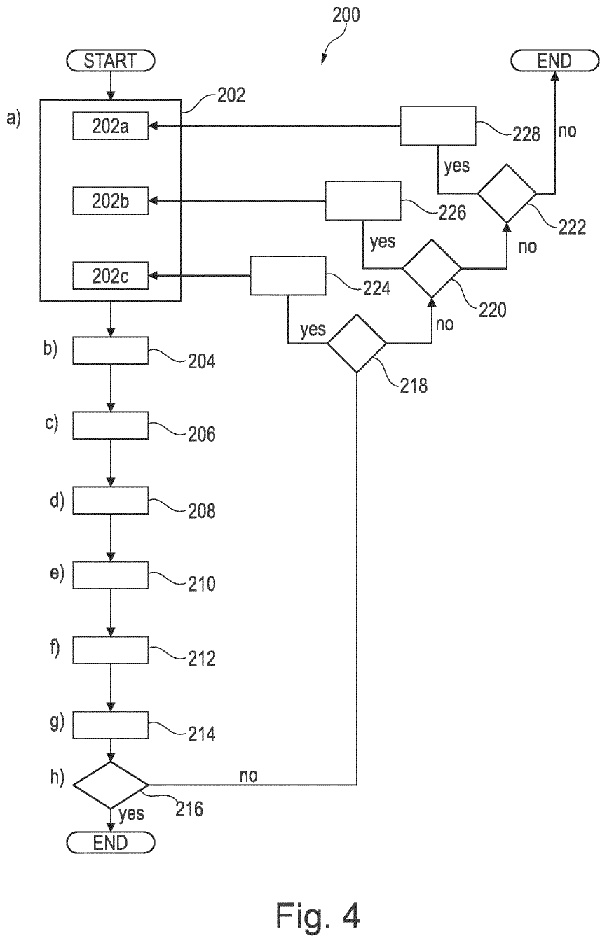

A flowchart representative of example machine readable instructions for implementing the apparatus 110 of FIGS. 1 and/or 2 is shown in FIG. 4. In this example, the machine readable instructions comprise a program for execution by a possessor, such as the processing unit 20 shown in the example apparatus in FIGS. 1 and/or 2. The program may be embodied in software stored on a tangible computer readable storage medium such as a CD-ROM, a floppy disk, a hard drive, a digital versatile disk (DVD), a Blu-ray disk, or a memory associated with the processing unit 20, but the entire program and/or parts thereof could alternatively be executed by a device other than the processing unit 20 and/or embodied in a firmware of dedicated hardware.

The program of FIG. 4 begins when a user input is received [step a), block 202] from one or more electronic devices as shown in FIG. 1. The user defines a dosage form (block 202a), e.g., via a graphical user interface of the illustrated example of FIG. 3A. The dosage form may comprise at least one of a capsule including a hard capsule and a soft capsule, a chewing gum, a cream, an emulsion including an emulsion concentrate and a microemulsion, a foam, a spray, a gel, a stick, granules, gummies, an implant, an ointment, a paste, pellets including coated pellets, a powder, a solution including an injection solution, a suppository, a suspension including a suspension concentrate, a sustained-release form, a tablet including a buccal tablet, a chewable tablet, a coated tablet, a detergent tablet, a dishwashing tablet, an effervescent tablet, a lozenge, an orodispersible tablet, and a vaginal tablet, and a therapeutic patch.

The user also enters a TPP (block 202b), e.g., via a graphical user interface of the illustrated example of FIG. 3C. Examples of the user-defined TPP may include:
user-defined TPP applicable to all kinds of dosage forms including, but not limited to, amount and/or concentration of the active ingredient; size, volume and/or weight of the dosage form; mechanical and/or rheological properties of the dosage form; release profile of the active ingredient; other application-relevant parameters; compatibility and stability; and other manufacturing-relevant properties.
user-defined TPP for solid oral dosage forms including, but not limited to, amount of AI per unit; size and/or weight of the dosage form; mechanical strength of the dosage form; desired release behaviour of the dosage form; disintegration time of the dosage form; dissolution profile of the AI; compatibility of active ingredients and excipients; probability to pass content uniformity criteria; flowability of a powder blend; tabletability of a powder blend; and compatibility and stability of active ingredients and excipients.
user-defined TPP for liquid and semi-solid dosage forms including, but not limited to, concentration of AI; volume of the dosage form; rheological behaviour and/or viscosity of the dosage form; spreading and/or adherence of the dosage form; dispersity and/or volume fractions of phases; hydrophilicity and/or lipophilicity; release behaviour of the dosage form; melting point of the dosage form; dissolution profile of the AI; and compatibility and stability of active ingredients and excipients.

The user also enters key physicochemical properties of an AI (block 202c), e.g., via a graphical user interface of the illustrated example of FIG. 3B. Examples of the key physicochemical properties of the AI may include at least one of: hydrophilicity and/or lipophilicity (e.g., distribution coefficient); melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactibility; hygroscopicity; water content (e.g., loss on drying); concentration of impurities; other chemical, physicochemical and/or physical properties; and information on compatibility and stability.

Next, the processing unit 30 of the example apparatus 110 of FIGS. 1 and/or 2 calculates key parameters of the AI relevant for the development of the dosage form based on the key physicochemical properties of the AI [step b), block 204]. The key parameters of the AI are calculated from the key physicochemical properties of the AI by normalizing and scaling the data.

For example, the two most important parameters in direct compression processes are flowability and tabletability of the powder. The flowability of powders can be characterized by measuring the angle of repose, Hausner ratio or flow rate through an orifice. Shear cell measurements, powder rheology and avalanche testing are more advanced tools to characterize powder flow. In contrast to flowability, the tabletability of powders is more difficult to assess. Measuring the resistance to crushing of tablets does not adequately describe the tabletability of a powder. And plotting the compressibility and compactibility profiles of a material is time-consuming.

To characterize the tabletability of a powder, a model may be used. To start the experiment, it may be needed to prepare tablets of the powder at five different compaction pressure levels.

Alternatively, if the powder is poorly compressible, it may be considered to prepare tablets of the powder in combination with a directly compressible excipient. In this case, the effect of the excipient is automatically subtracted by the processing unit 30. In the next step, the user measures the weight of the prepared tablets, their thickness and diameter, and their breaking force. The data may be entered via a command line interface or a graphical user interface. In the last step, the processing unit 30 calculates the porosity and tensile strength of the tablets. A modified version of the Gurnham equation may be used to model the porosity of the tablet as a function of the applied pressure [see G. K. Reynolds, J. I. Campbell, R. J. Roberts, A compressibility based model for predicting the tensile strength of directly compressed pharmaceutical powder mixtures, International Journal of Pharmaceutics, 531 (2017) 215-224]:

InP=InP₀−k_{c}·ε(P)

The Ryshkewitch-Duckworth equation may be used to describe the change in tensile strength with changing density of the tablet:

InT=InT₀−k_{b}·ε(P)

Once all data has been entered, the processing unit 30 calculates the compaction pressure at zero porosity, the compressibility resistance, the tensile strength at zero porosity and the bonding capacity. These four values are used to evaluate the tabletability of the powder.

To estimate the processability of powders, different parameters may be considered. The parameters may be grouped into the categories such as: Particle size, powder density, flowability and tabletability. All parameters may be scaled from 0 to 10, where 0 means “insufficient” and 10 means “excellent”; 5 is the acceptance value for direct compression. The chosen parameters and their limits build on the Manufacturing Classification System [see M. Leane, K. Pitt, G. Reynolds, A proposal for a drug product Manufacturing Classification System (MCS) for oral solid dosage forms, Pharmaceutical Development and Technology, 20 (2015) 12-21]. Using the angle of repose as an example, a value of 65° is normalized as 0; a value of 45° is normalized as 5; and a value of 25° is normalized as 10.

For example, fourteen parameters (i.e., dl 0 value, d50 value, d90 value, distribution span, bulk density, tapped density, compressibility index, Hausner ratio, angle of repose, compaction pressure at a porosity of 0.15, tensile strength at a porosity of 0.15, tensile strength at 100 MPa compaction pressure, tensile strength at 150 MPa compaction pressure and tensile strength at 250 MPa compaction pressure) may be used to evaluate the processability of the active ingredient. All parameters may be scaled from 0 to 10, where 0 means “insufficient” and 10 means “excellent”; 5 is the acceptance value for direct compression.

The parameters may be grouped into the following categories: processability (mean value of all parameters), particle size (mean value of d10 value, d50 value, d90 value and distribution span), powder density (mean value of bulk and tapped density), powder flow (mean value of compressibility index, Hausner ratio and angle of repose) and tabletability (mean value of compaction pressure at a porosity of 0.15, tensile strength at a porosity of 0.15, tensile strength at 100 MPa compaction pressure, tensile strength at 150 MPa compaction pressure and tensile strength at 250 MPa compaction pressure).

A radar chart may be used to quickly identify the strengths (values greater than or equal to 5) and weaknesses (values less than 5) of an active ingredient. In general, it can be concluded that the processability is improving, as the area circumscribed by the curve is increasing. Direct compression is possible if the parameters processability, powder flow and tabletability are greater than or equal to 5; dry granulation (e.g., roller compaction, slugging etc.) is possible if the tabletability parameter is greater than or equal to 5; wet granulation (e.g., fluid-bed or high-shear granulation) is always possible. An example for the calculated key parameters of the AI is illustrated in FIG. 3D.

Optionally, the probability of passing a content uniformity test may be checked. The content uniformity of pharmaceutical dosage forms can be affected by the particle size and size distribution of the active ingredient. With the described apparatus, it is possible to set particle size limits for a given dose and eliminate problems of content uniformity associated with particle size. A modified version of the Yalkowsky-Bolton equation may be used to calculate the relative standard deviation of the dose for a given particle size distribution [see B. R. Rohrs, G. E. Amidon, R. H. Meury, P. J. Secreast, N. M. King, C. J. Skoug, Particle size limits to meet USP content uniformity criteria for tablets and capsules, Journal of Pharmaceutical Sciences, 95 (2006) 1049-1059]. The theory assumes homogeneous mixing and that the particle size distribution is log-normal.

A diagram, such as the diagram shown in FIG. 3E, may indicate the maximum volume median particle diameter (d50 value) predicted to pass the content uniformity test at stage I with the given confidence level (p-value) as a function of the distribution width (d90/d50). The diagram can be used to estimate the necessary particle size to ensure content uniformity criteria are met. The diagram also demonstrates that the maximum acceptable d50 value increases significantly, if the width of the particle size distribution is reduced. For example, by narrowing the distribution width (d90/d50) from 4 to 2, the acceptable d50 value increases about fourfold. Since larger particles impact content uniformity to a much greater extent, eliminating large particles (e.g., by sieving) can significantly alter the required particle size.

Then, the processing unit 30 predicts the key physicochemical properties of the AI when combined with the one or more excipients selected from an excipient database by applying mixing rules. The apparatus subsequently processes the data and re-calculates the key parameters of the AI when combined with the one or more excipients selected from an excipient database by normalizing and scaling the data [step c), block 206]. A user may select one or more excipients via an example graphical user interface of FIG. 3F. The re-calculated key parameters may be compared with the previously calculated key parameters to determine whether deficient properties of the API can be corrected with the excipient. For example, a radar chart as illustrated in FIG. 3G may be used to demonstrate the improvements.

In other words, the apparatus predicts the processability of powder blends (i.e., combinations of an active ingredient with a filler/binder). The user may need to enter the properties of the active ingredient (or select an active ingredient from a database), select an excipient or excipient blend from the list, and enter the weight fraction of the active ingredient.

For example, the properties of the powder blend (i.e., particle size distribution, bulk density, tapped density, angle of repose, compressibility and compactibility profile) are estimated from single-component data by applying mixing rules. To estimate the particle size distribution of the powder blend, the cumulative size distributions of the individual components are reconstructed from their d10, d50 and d90 values (it is assumed that the particles are spherical and log-normally distributed). The cumulative size distribution of the powder blend is then derived from the volume-weighted arithmetic mean of the individual curves.

The bulk density of the powder blend is calculated from the weighted arithmetic mean of the individual values; more weight is given to the component with the smaller bulk density. The tapped density of the powder blend is calculated from the weighted arithmetic mean of the individual values; more weight is given to the component with the larger tapped density. The angle of repose of the powder blend is calculated from the weighted arithmetic mean of the individual values; more weight is given to the component with the larger angle of repose.

The compressibility profile of the powder blend is derived from the volume-weighted arithmetic mean of the individual profiles; a least squares fitting is done to determine the compaction pressure at zero porosity and compressibility resistance. The compactibility profile of the powder blend is derived from the volume-weighted geometric mean of the individual profiles; a least squares fitting is done to determine the tensile strength at zero porosity and bonding capacity.

Next, the processing unit 30 of the example apparatus 110 of FIGS. 1 and/or 2 selects at least one promising excipient from the one or more selected excipients capable of improving the key parameters of the AI [step d), block 208], and suggests a manufacturing process based on the AI, the at least one selected promising excipient, and the dosage form [step e), block 210]. The promising excipients are those which can better correct the deficient properties of the API in comparison with other excipients. FIG. 3G illustrates an example of the promising excipient, i.e. Ludipress®, which can correct the deficient properties of the AI such that the formulation is suitable for direct compression.

For example, the apparatus calculates the weight fraction of the active ingredient based on the given dose and tablet weight; 7% are subtracted from the tablet weight to accommodate the disintegrant and lubricant. In the next step, the apparatus predicts the processability of all possible active ingredient-excipient combinations. The properties of the powder blends (i.e., particle size distribution, bulk density, tapped density, angle of repose, compressibility and compactibility profile) are estimated from single-component data by applying mixing rules.

Thus, fourteen parameters (i.e., d10 value, d50 value, d90 value, distribution span, bulk density, tapped density, compressibility index, Hausner ratio, angle of repose, compaction pressure at a porosity of 0.15, tensile strength at a porosity of 0.15, tensile strength at 100 MPa compaction pressure, tensile strength at 150 MPa compaction pressure and tensile strength at 250 MPa compaction pressure) may be used to evaluate the processability of the active ingredient. All parameters may be scaled from 0 to 10, where 0 means “insufficient” and 10 means “excellent”; 5 is the acceptance value for direct compression.

The parameters may be grouped into the following categories: processability (mean value of all parameters), particle size (mean value of d10 value, d50 value, d90 value and distribution span), powder density (mean value of bulk and tapped density), powder flow (mean value of compressibility index, Hausner ratio and angle of repose) and tabletability (mean value of compaction pressure at a porosity of 0.15, tensile strength at a porosity of 0.15, tensile strength at 100 MPa compaction pressure, tensile strength at 150 MPa compaction pressure and tensile strength at 250 MPa compaction pressure).

The radar chart can also be used to quickly identify the strengths (values greater than or equal to 5) and weaknesses (values less than 5) of a powder blend. In general, it can be concluded that the processability is improving, as the area circumscribed by the curve is increasing. Direct compression is possible if the parameters processability, powder flow and tabletability are greater than or equal to 5; dry granulation (e.g., roller compaction, slugging etc.) is possible if the tabletability parameter is greater than or equal to 5; wet granulation (e.g., fluid-bed or high-shear granulation) is always possible.

Once all combinations have been tested, the apparatus sorts the tested excipients and excipient blends according to their performance. For this purpose, a weighted mean of all 14 parameters is calculated. More weight is given to tabletability and powder flow; particle size and powder density are less important. Next, the apparatus suggests a suitable manufacturing process and selects the most relevant excipient or excipient blend.

Using direct compression as an example, according to the Manufacturing Classification system, direct compression is feasible if the material meets certain requirements regarding particle size and shape, blend uniformity, powder flow, powder density, tableting performance, and mechanical strength of the compact.

For example, in direct compression processes, excipients and excipient blends with insufficient flowability and/or tabletability (values less than 5) are sorted out. All excipients and excipient blends are rated on a scale from zero (“not qualified”) to five stars (“most qualified”). “Five stars” denotes excipients or excipient blends with the highest performance; “one star” denotes excipients or excipient blends with the lowest performance; “zero stars” means that the excipient or excipient blend is not qualified for direct compression processes.

Then, the apparatus calculates a starting formulation with the selected filler-binder combination. A superdisintegrant is added; the amount depends on the selected filler-binder combination. For example, if the filler-binder combination already contains a disintegrant (e.g., ready-to-use excipients), the amount of superdisintegrant is reduced accordingly. If the tabletability of the powder blend is medium (value less than 6), a superdisintegrant with binding properties is chosen (e.g., Kollidon® CL-SF); otherwise, a regular superdisintegrant (e.g., Kollidon® CL-F) is added. Sodium stearyl fumarate is added as a lubricant; the amount depends on the selected filler-binder combination. In contrast to magnesium stearate, sodium stearyl fumarate does not cause overlubrication; it shows less incompatibilities with active ingredients. Once the correct amounts of superdisintegrant and lubricant have been determined, the amount of the filler-binder combination is recalculated.

Then, the processing unit 30 of the example apparatus 110 of FIGS. 1 and/or 2 predicts product properties based on the suggested manufacturing process, a combination of the AI and the at least one selected promising excipient, and the dosage form [step f), block 212] and determines whether the predicted product properties comply with the user-defined TPP [step g), block 214].

If it is determined that the predicted product properties comply with the user-defined TPP, e.g. falling within a predefined range of the user-defined TPP, a suitable formulation can be identified based on the combination of the AI and the at least one selected promising excipient, the suggested manufacturing process, and the dosage form [step f), block 216]. An example of the identified formulation is illustrated in FIG. 3H. In step 216, if at least one formulation has been identified, the user may prepare the formulation in the laboratory and characterize the obtained product. The development process is finished if the experimental results (e.g., content uniformity, dissolution profile, mechanical strength of tablet etc.) comply with the target profile. Otherwise, the apparatus may suggest optimizing the properties of the AI, adjusting the TPP or selecting a different dosage form.

If it is determined that the predicted product properties do not comply with the user-defined TPP, the processing unit 30 of the example apparatus 110 may suggest optimizing the properties of the AI (block 218) with at least one additional technological measure, based on a difference between the predicted product properties and the user-defined TPP. The at least one additional technical measure comprises at least one of: milling or micronization, and addition of and processing with excipients. Alternatively or additionally, the processing unit 30 may suggest adjusting the TPP (block 220) or selecting a different dosage form (block 222) based on a difference between the predicted product properties and the user-defined TPP. Then, the user has the possibility to optimize the properties of the AI (block 224), re-define the TPP (block 226), and/or re-select a dosage form (block 228). Then, the processing unit 30 performs again steps b) to h), until a suitable formulation has been identified with the product properties complying with the user-defined or user-redefined TPP, and the program of FIG. 4 ends. The same procedure (blocks 218-228) may also apply when the experimental results do not comply with the target profile.

As an option, the user has the possibility to print the formulation, download relevant information (e.g., quality, regulation, and technical documents), and order product samples. In addition, the user may have the possibility to provide feedback (e.g., regarding usability, information content, formulation outcome, etc.), which is used to improve the user experience and the system itself.

The system may additionally contain a compilation of examples of final products incl. their recipes, manufacturing technologies and properties. This compilation can be accessed by the user, e.g. by searching for the active, the dose and the dosage form. The manufacturability and the properties of these drug products have been experimentally proven already.

In another exemplary embodiment of the present invention, a computer program or a computer program element is provided that is characterized by being adapted to execute the method steps of the method according to one of the preceding embodiments, on an appropriate system. The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims. In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A computer-implemented method (200) for identifying a suitable formulation for product development, comprising:
a) receiving (210), via an input channel, a user input that defines:
a dosage form;
a target product profile, TPP, comprising a minimum product requirement; and
key physicochemical properties of an active ingredient, AI;
b) calculating (220), by a processor, key parameters of the AI relevant for the development of the dosage form based on the key physicochemical properties of the AI;
c) predicting (230), by the processor, the key parameters of the AI when combined with the one or more excipients selected from an excipient database by applying mixing rules;
d) identifying (240), by the processor, at least one promising excipient from the one or more selected excipients capable of improving the key parameters of the AI;
e) suggesting (250), by the processor, a manufacturing process based on the AI, the at least one selected promising excipient, and the dosage form;
f) predicting (260), by the processor, product properties based on the suggested manufacturing process, a combination of the AI and the at least one selected promising excipient, and the dosage form;
g) determining (270), by the processor, whether the predicted product properties comply with the user-defined TPP; and
h) identifying (280), by the processor, a suitable formulation based on the combination of the AI and the at least one selected promising excipient, the suggested manufacturing process, and the dosage form, if it is determined that the predicted product properties comply with the user-defined TPP.

2. The computer-implemented method according to claim 1, further comprising:
if it is determined that the predicted product properties do not comply with the user-defined TPP or if it is determined that an experimental result obtained after preparing and characterizing the identified suitable formulation does not comply with the user defined TPP, performing at least one of the following steps:
suggesting at least one additional technological measure to optimize the key parameters of the AI, based on a difference between the predicted product properties and the user-defined TPP or a difference between the experimental result and the user-defined TPP;
suggesting to adjust the user-defined TPP based on a difference between the predicted product properties and the user-defined TPP or a difference between the experimental result and the user-defined TPP; and
suggesting to select a different dosage form based on a difference between the predicted product properties and the user-defined TPP or a difference between the experimental result and the user-defined TPP.

3. The computer-implemented method according to claim 2,
wherein the at least one additional technical measure comprises at least one of:
milling or micronization; and
addition of and processing with excipients.

4. The computer-implemented method according to claims 2 to 3, further comprising:
if the predicted product properties do not comply with the user-defined TPP or if it is determined that the experimental result obtained after preparing and characterizing the identified suitable formulation does not comply with the user defined TPP,
repeatedly performing a sequence comprising:
receiving a further user input related to a different dosage form, a user-redefined TPP, and/or re-determined key physicochemical properties of the AI; and
performing steps b) to h), until a suitable formulation has been identified with the product properties complying with the user-defined or user-redefined TPP.

5. The computer-implemented method according to claim 1,
wherein the product development comprises at least one of the following:
development of cleaning agents;
development of cosmetic products;
development of dietary supplements;
development of drug products;
development of fungicide formulations;
development of herbicide formulations;
development of pesticide formulations; and
development of washing agents.

6. The computer-implemented method according to claim 1,
wherein the dosage form comprises at least one of a capsule, a chewing gum, a cream, an emulsion a foam, a spray, a gel, a stick, granules, gummies, an implant, an ointment, a paste, pellets, a powder, a solution, a suppository, a suspension, a sustained-release form, a tablet, and a therapeutic patch.

7. The computer-implemented method according to claim 1,
wherein the user-defined TPP comprises at least one of:
amount and/or concentration of the active ingredient;
size, volume and/or weight of the dosage form;
mechanical and/or rheological properties of the dosage form;
release profile of the active ingredient;
other application-relevant parameters;
compatibility and stability; and
other manufacturing-relevant properties.

8. The computer-implemented method according to claim 7,
wherein the user-defined TPP comprises at least one of:
amount of AI per unit;
size and/or weight of the dosage form;
mechanical strength of the dosage form;
desired release behaviour of the dosage form;
disintegration time of the dosage form;
dissolution profile of the AI;
compatibility of active ingredients and excipients;
probability to pass content uniformity criteria;
flowability of a powder blend;
tabletability of a powder blend; and
compatibility and stability of active ingredients and excipients.

9. The computer-implemented method according to claim 7,
wherein the user-defined TPP comprises at least one of:
concentration of AI;
volume of the dosage form;
rheological behaviour and/or viscosity of the dosage form;
spreading and/or adherence of the dosage form;
dispersity and/or volume fractions of phases;
hydrophilicity and/or lipophilicity;
release behaviour of the dosage form;
melting point of the dosage form;
dissolution profile of the AI; and
compatibility and stability of active ingredients and excipients.

10. The computer-implemented method according to claim 1,
wherein the key physicochemical properties of the AI comprise at least one of:
hydrophilicity and/or lipophilicity (e.g., distribution coefficient);
melting point;
permeability across biological or artificial lipid membranes;
solubility in water, solvents, co-solvents and/or biorelevant media;
miscibility with water, solvents, co-solvents and/or biorelevant media;
true density;
viscosity;
wettability;
interfacial and/or surface tension;
particle size distribution data;
particle morphology, shape and/or aspect ratio;
bulk and tapped density;
flowability;
compressibility and compactibility;
hygroscopicity;
water content;
concentration of impurities;
other chemical, physicochemical and/or physical properties; and
information on compatibility and stability.

11. The computer-implemented method according to claim 1,
wherein the user-defined TPP comprises a dose of AI per unit and a maximum weight of the dosage form;
wherein step c) further comprises:
calculating weight fractions of the AI and the one or more excipients selected from the excipient database based on the dose of AI per unit and the maximum weight of the dosage form;
predicting properties of a combination of the AI and the one or more excipients; and
wherein step d) further comprises selecting at least one promising excipient from the one or more excipients if the properties of a corresponding mixture satisfy a predefined criterion.

12. The computer-implemented method according to claim 1,
wherein the dosage form comprises a pharmaceutical dosage form.

13. An apparatus (110) for identifying a suitable formulation for product development, comprising:
an input unit (10); and
a processing unit (20) configured to:
a) receive a user input, via the input unit, wherein the user input defines:
a dosage form;
a target product profile, TPP, comprising a minimum product requirement; and
key physicochemical properties of an active ingredient, AI;
b) calculate key parameters of the AI relevant for the development of the dosage form based on the key physicochemical properties of the AI;
c) re-calculate the key parameters of the AI when combined with the one or more excipients selected from an excipient database;
d) select at least one promising excipient from the one or more selected excipients capable of improving the key parameters of the AI;
e) suggest a manufacturing process based on the AI, the at least one selected promising excipient, and the dosage form;
f) predict product properties based on the suggested manufacturing process, a combination of the AI and the at least one selected promising excipient, and the dosage form;
g) determine whether the predicted product properties comply with the user-defined TPP; and
h) identify a suitable formulation based on the combination of the AI and the at least one selected promising excipient, the suggested manufacturing process, and the dosage form, if it is determined that the predicted product properties comply with the user-defined TPP.

14. A system (100) for identifying a suitable formulation for product development, comprising:
an apparatus (110) according to claim 12; and
a web server (140) configured to interface with a user via a webpage and/or an application program served by the web server;
wherein the apparatus is configured to provide a graphical user interface, GUI, to a user, by the webpage and/or the application program.

15. A computer program element comprising sets of instructions, wherein, when the sets of instructions are executed on a processor of an apparatus, the sets of instructions cause the apparatus or the system to perform the method of claim 1.
